(19) United States
(12) Patent Application Publication
(10) Pub. No.: US 2004/0228795 A1
(43) Pub. Date: Nov. 18, 2004

(54) Method for producing anterior ischemic optic neuropathy in animals

(76) Inventor: Steven L. Bernstein, Chevy Chase, MD (US)

Correspondence Address: Rochelle Lieberman, Esq., Lieberman & Brandsdorfer, LLC, 12221 McDonald Chapel Drive, Gaithersburg, MD, 20878, US

(21) Appl. No.: 10/868,600

(22) Filed: Jun. 15, 2004

Related U.S. Application Data
(63) Continuation of application No. 09/727,603, filed on Feb. 7, 2001.

(51) Int. Cl. A61K 49/00; A61K 31/555; A61N 1/30

(52) U.S. Cl. 424/9.2; 514/185; 604/20

(57) ABSTRACT

Anterior ischemic optic neuropathy is an ischemic disease affecting the optic nerve. A blockage of vessels supplying the intra-retinal portion of the optic nerve causes loss of axon transport stasis, retinal ganglion cell, specific dysfunction, and RGC death. Research has been limited by the lack of low-cost models for this disease. Using a custom contact lens, an intravenous injection of photosensitizing agent anesthetizes male Sprague-Dawley rats. A laser activate dye within the small vessels perfusing the optic nerve. This treatment spared the larger vessels perfusing the inner retina. Electropliysiologically, a decrease in amplitude of the visual evoked potential is noted. Histologically, alterations in axonal transport are seen. Reverse-transcriptase based Polymerase chain later retinal gene expression changes in treated animals. This method replicates many cellular and molecular level changes in a low-cost animal model.

### FIELD OF THE INVENTION

The present invention relates to a method to determine the specific genes involved in damage to the cells making Lip the axon fibers of the optic nerve, and more particularly, to determine the changes in retinal ganglion cell expression that occur in glaucoma and ischemic optic neuropathy.

### BACKGROUND OF THE INVENTION

Glaucoma and ischemic optic neuropathy are leading causes of blindness in the United States, yet despite the known histology and mechanics of the disease (ischemic damage to the optic nerve, which includes the axons of the retinal ganglion cells that synaptically connect the retina ganglion to the central nervous system), the specific genes involved in optic nerve maintenance and axonal transport of factors supporting optic nerve function are largely unknown. Glaucoma and ischemic optic neuropathy are painless diseases, which involve damage to the optic nerve and a chronic optic netiropathy. Research has shown that heat shock Protein 90 (HSP90), a chaperone protein has been shown to be intimately involved in retinal ganglion cell (RGC) function, is present in high levels in the optic nerve; the key site of glaucomatous and optic neuropathic damage. Expression of chaperone proteins have been associated with enhanced protection of retinal ganglion cells, that are subjected to stresses similar to those found in ischeinic optic nerve disease.

The use of this invention is to enable the examination in vivo of the changes in retinal ganglion cell gene expression that occur in glaucoma and optic neuropathy, using a low cost (rodent) animal model. There has recently been developed a new model of optic neuropathy in rat, which utilizes photosensitizing agents and irradiation of the optic nerve.

The chronic optic neuropathies selectively affect the retinal ganglion cell and its axon in an intact animal. Intact, adult, normal retinal ganglion cell function is part of a complex system consisting of full length, normal, differentiated retinal ganglion cells with their cell bodies in the retina, and their longest cellular extensions (axons) synapsing in the central nervous system. The blood supply of the individual components (RGC body, axon, CNS tennination) are also separate. Intact adult RGC's cannot be isolated or maintained in culture. Thus, the intact adult retinal ganglion cell function cannot be studied in cell culture since it is part of a complex system. The majority of glaucoma studies and neuropathy studies have been conducted in rhesus monkeys, requiring the expenditure of large amounts of money and the sacrifice of previous primates, as well as raising ethical considerations of primate usage. A new model of optic neuropathy in a rat, utilizing retinal laser treatment and photosensitization has been developed in the present invention. The new model produces a potentially reversible, ischeinic optic neuropathy. This method differs from the previous methods in that it is potentially reversible, is not induced by manual (incisional) surgery, and is variable in the level of severity that can be produced. The new model utilizes rat, and thus partially obviates the need for higher species (i.e. primate) in studying optic neuropathy and some forms of glaucoma.

### BRIEF DESCRIPTION OF THE DRAWING

FIG. 1 depicts the area of the eye affected by the laser and the effect produced by laser irradiation.

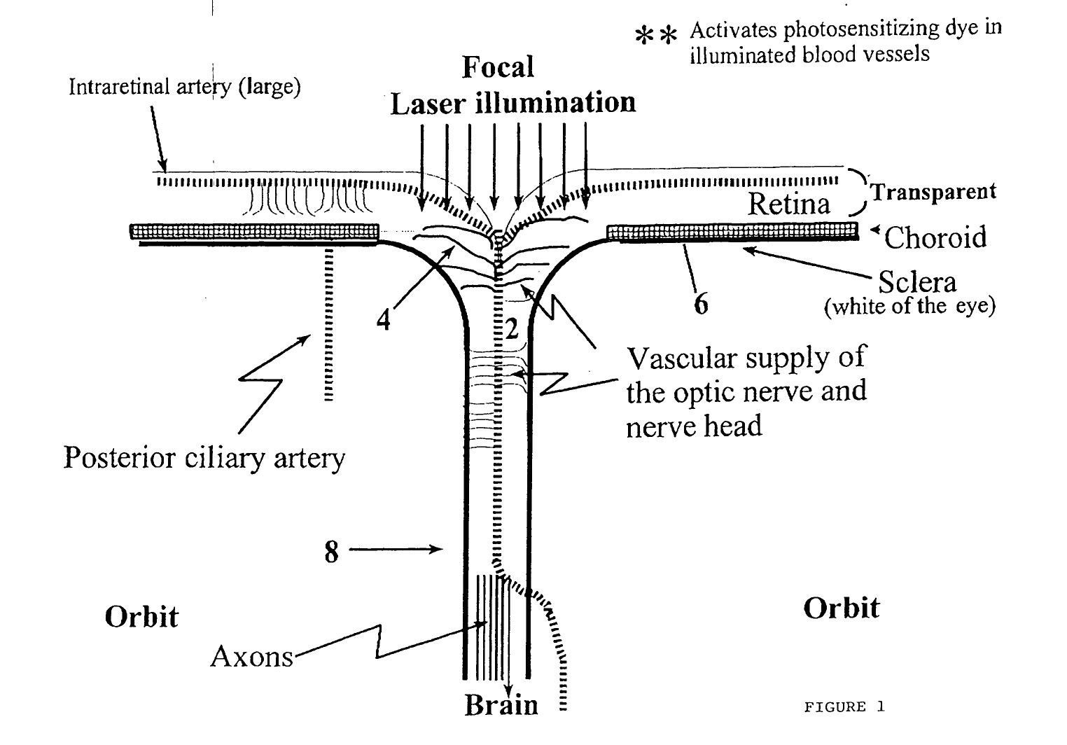

### DETAILED DESCRIPTION OF THE INVENTION

Referring specifically to the single FIGURE, optic nerve 2 conveys visual information from the retina 4 to the brain for interpretation. Numeral 6 designates the sclera.

Rat (Rattus Norvegicus) are suitably anesthetized (typically with ketamine 35 mg/kg and xylazine 5 mg/kg) and the pupils of the eyes are dilated with an appropriate agent. After anesthesia, an intravenous injection of photosensitizing agent (in this case, Rose bengal, a fluorinated derivative of fluorescein) is administered through the tail vein. The choice of photosensitizing agent is based on the ability to generate free radical oxygenions on illumination. Dosed administration of the agent is typically given based on the mg/kg animal weight. The basis of the effect is not heat generation at the laser site, but rather a function of the effect that oxygen radicals have on damaging blood vessel endothelium inducing thrombosis and closure of the small vessels (capillaries) supplying the optic nerve head; at the junction of the retina and optic nerve.

One minute after the injection, a retinal fundus (standard opthamalic connotation for the retinal surface) contact lens that I have developed, that stabilizes the eye and allows direct visualization and photic treatment of the rat retina is applied to one eye, and the optics nerve is treated using a low intensity laser light; either argon laser light at 510 nm to 565 nm laser light with frequencies between 510 nm and 565 nm, or frequency double ND:YAG laser light at 535 nm, a spot size as determined by the specimen, but nominally 500 microns size (must cover the optic nerve); laser power output less than 0.010 mW. A variety of laser frequencies, depending on the absorptive and emission spectrum of the dye compound may be used to generate oxygen free radicals. The treatment is continued for a suitable length of time to produce the desired extent of vessel damage and ischeinic typically in the range of 8-40 seconds. Longer treatment (>25 seconds) is associated with central retinal artery blockage (retinal stroke).

One day after treatment, optic nerve ischemic can be visually identified by swelling of the optic nerve head. Using electrophysiological means (visual evoked potential), optic nerve functional loss can be measure by a loss of VEP amplitude from 20-100%. A measure of the selectivity of the coupled photosensitizing agent—laser treatment in producing optic neuropathy can be determined by laser illumination of the optic nerve in animals that are not injected with photosensitizing agent. Typically the control eyes show little or no change in gross features, histological features, and electrophysiological (VEP) measures.

Animal VEP's are performed using standard electrophysiological techniques. The head of the animal is shaved and electrode paste is applied to two sites on the animals head. Electrical transmission along the optic nerve is measured and amplified. After eliciting the VEP, descriptive statistical analysis is used to compare the results of the VEP tests. Ocular tissues of the treated and control animals are also examined histologically.

### BRIEF DESCRIPTION OF THE PRESENT INVENTION

In accordance with the present invention, recently that has been developed a new model of optic neuropathy in rat utilizing retinal laser treatment combined with photochemical-induced closure of selected vessels (capillaries) of the optic nerve. This model is useful in studying in vivo optic neuropathy and the physiological responses to neuropathy in the intact organism.

The treatment contemplated herein damages only small capillaries and the purpose of the treatment is described above. Specifically, the treatment is the exposure to laser light of the optic nerve of the specimen. The exposure as indicated above is to affect only the capillaries supplying the optic nerve. Ancillary damage must be minimized.

The result of the above is that there is now a model that grossly and histologically resembles anterior, ischemic optic neuropathy that looks like the human optic nerve disease encountered in anterior, ischemic optic neuropathy, glaucoma, and related eye diseases. There is a block of much of optic nerve transport one day after a single optic nerve vessel treatment. After awhile, about 40% of retinal transport cells dies. Experiments can now be conducted on the rat to attempt to locate the specific genes involved so they can be treated to cure such diseases, and in particular, anterior ischemic optic neuropathy and glaucoma. The experiments concern an attempt to prevent occurrence of severe damage to the optic nerve.

Once given the above disclosure, many other features, modifications and improvements will become apparent to the skilled artisan. Such features, modifications and improvements are, therefore, considered to be part of this invention, the scope of which is to be determined by the following claims.

What is claimed is:

1. A method of preparing an animal for use as a specimen in ischemic optic nerve disease research comprising:
employing photosensitizing agents injected into the body of the specimen, and
irradiating the optic nerve with energy from a laser to damage capillaries feeding the anterior portion of the optic nerve.

2. The method of preparing an animal according to claim 1, wherein for rats the laser for irradiating the optic nerve has approximately a 500 to 600 micron size spot at 50 microwatts.

3. The method of preparing an animal according to claim 2, wherein the spot is directed to the optic nerve for approximately 7 to 30 seconds determined primarily by the retinal artery occlusion desired and under standard conditions, preferably 15 seconds.

4. The method of preparing an animal according to claim 1, wherein the laser is an argon green laser.

5. The method of preparing an animal according to claim 1, wherein the laser is a frequency double YAG laser.

6. The method of preparing an animal according to claim 1, wherein the laser is an yttrium aluminum garnet negdym material also known as YAG.
